(19) United States
(12) Patent Application Publication
(10) Pub. No.: US 2024/0415992 A1
(43) Pub. Date: Dec. 19, 2024

(54) DISINFECTING A CONTAINER PROCESSING SYSTEM

(71) Applicant: KRONES AG, Neutraubling (DE)

(72) Inventors: Torsten KILGENSTEIN, Geisenfeld (DE); Erwin KNIELING, Pfatter (DE)

(21) Appl. No.: 18/703,926

(22) PCT Filed: Oct. 26, 2022
(86) PCT No.: PCT/EP2022/079978

§ 371 Date: Apr. 23, 2024

(30) Foreign Application Priority Data
Oct. 29, 2021 (DE) 102021128275.6

(51) Int. Cl. A61L 2/08; A61L 2/24; B01J 21/06; B01J 35/39; B67C 3/00

(52) U.S. Cl. CPC A61L 2/088; A61L 2/24; B01J 21/063; B01J 35/39; B67C 3/005

(57) ABSTRACT

The invention relates, inter alia, to a container processing system for processing containers, preferably bottles or cans. The container processing system comprises at least one component having a surface with a material that can be photoactivated for the disinfecting of the surface. Advantageously, portions of the container processing system that are relevant to hygiene can be self-sterilizing.

### TECHNICAL FIELD

The invention relates to a container processing system. The invention also relates to a method for treating a container processing system.

### TECHNICAL BACKGROUND

In a container processing system, containers can be transported through the individual system parts and, for example, be filled and closed. During processing and transport, the containers come into contact with a plurality of surfaces of the container processing system.

For surface sterilization, the surfaces of the container processing system can be treated with hot caustic solution or foam cleaning agent or by manual cleaning, for example at the filling machine or the rinser. Surface sterilization can also be accomplished by disassembly and manual cleaning, for example at the blow molding machine, the preform feed, the closure feed and the container feed.

Transport chains and machine surfaces or faces which are in contact with the container can be cleaned, for example, by means of acidic or alkaline cleaners, foam cleaning and high-pressure water in order to prevent colonization by organisms which could have a detrimental effect on the products to be introduced into containers.

Disadvantages of the cleaning methods described above can be the high frequency of the required cleaning, the need to shut down the machines, and high costs.

A further disadvantage can be that methods which have a germicidal effect can also have a negative effect on the product to be introduced into containers. A time-consuming and proper flushing can therefore be necessary. The degree of cleanliness can also decrease over time.

The invention is based on the object of creating an improved technique for disinfecting a container processing system.

### SUMMARY OF THE INVENTION

The object is achieved by the features of independent claim 1. Advantageous developments are specified in the dependent claims and the description.

One aspect of the present disclosure relates to a container processing system for treating containers, preferably bottles or cans. The container processing system comprises at least one component having a surface with a material that can be photoactivated for the disinfecting (e.g., killing or neutralizing of germs, such as bacteria, viruses, spores, fungi) of the surface.

Advantageously, at least portions of the container processing system that are relevant to hygiene can thus be self-sterilizing. The surface can, so to speak, disinfect itself by means of the photoactivatable material when daylight or lamp light hits the surface. The risk of contamination of the containers or of the contents in the containers can thus be significantly reduced. In addition, cleaning intervals for manual cleaning, for example by means of foam, hot caustic solution, etc., can at least be extended. There are fewer machine stoppages for cleaning, so that overall availability of the system can be increased. The photoactivatable material can be harmless for food and also can only have an effect on the surroundings directly adjacent to the surface, so that a negative effect on the product introduced into containers can be prevented.

In one exemplary embodiment, the material is a photocatalyst material and/or the material comprises titanium dioxide.

In another exemplary embodiment, the material, when irradiated with light, is stimulated to transfer energy to oxygen in the surroundings of the surface, so that singlet oxygen is formed for disinfecting the surface. Alternatively or additionally, the material, when irradiated with light, can form a singlet oxygen layer (e.g., adjacent to the surface) for disinfecting the surface. Advantageously, the singlet oxygen can kill and neutralize germs as an oxidizing agent by oxidative degeneration.

In another exemplary embodiment, the at least one component has a user interface, an operating element, a preferably touch-sensitive display, a keyboard, an emergency-off button, a safety switch, and/or a door opener. Thus, germ deposits can advantageously be prevented on frequently touched operating surfaces.

In another exemplary embodiment, the surface of the at least one component is a container contact surface for contacting containers during operation of the container processing system. Advantageously, it can thus be made possible that the container contact surface disinfects itself, so that contamination of the containers or of the product introduced therein can be prevented.

In another exemplary embodiment, the at least one component has a guide element for guiding the containers. The guide element can preferably be a (e.g., rod-shaped) guide railing, a guide wall, a guide format part adapted to a geometry of the containers to be processed, or a wear strip of a container guide. Advantageously, it can thus be prevented that the containers or their contents are contaminated by the guide element. In particular, the use as a wear strip is advantageous since the wear strip is replaced at regular intervals anyway and thus a possible weakening of the effect of the self-sterilization over time can be counteracted.

In another exemplary embodiment, the at least one component comprises a transport element for transporting the containers. The transport element can preferably be a transport chain, a conveyor belt, a transport mat, a container clamp or a container gripper. Alternatively or additionally, the at least one component comprises a container conveyor for conveying the containers, preferably a transport starwheel or a linear conveyor. Advantageously, it can thus be prevented that the containers or their contents are contaminated by the transport element or the container conveyor.

In another exemplary embodiment, the at least one component is arranged in a portion of the container processing system between a rinsing device of the container processing system and a filling device of the container processing system. Advantageously, recontamination of the containers on the way to the filling process after the rinsing process can thus be prevented.

The rinsing device can preferably be arranged upstream of the filling device (with respect to the container stream).

For example, a container conveyor can connect the rinsing device and the filling device to one another. The at least one component preferably comprises this container conveyor.

It is also possible for the at least one component to comprise a container conveyor which connects a filling device and a closing device of the container processing system to one another. Thus, contamination of the containers which have already been filled but have not yet been closed can advantageously be prevented.

In one embodiment, the at least one component comprises (at least) one container processing device for processing the containers, preferably a heating device for heating preforms for producing the containers, a container production device (e.g., blow-molding device) for producing the containers, a coating device for (e.g., internally) coating the containers, a rinsing device for rinsing the containers, a filling device (e.g., filler carousel) for filling the containers, a closing device for closing the containers, an inspection device for inspecting the containers, a labeling device for labeling the containers, a printing device for printing on the containers, and/or a packaging device for packaging the containers.

In one embodiment, the surface is formed by a spray-applied (sprayed-on) coating having the material. The coating can preferably be spray-applied or sprayed on in a comparatively short time in a simple manner. It is therefore not necessary, for example, to modify the production of the at least one component in a complex manner so that the surface has the material. Advantageously, the coating can be renewed at regular intervals, for example, so as not to risk a diminishing effect of the disinfection.

In another embodiment, the photoactivatable material is mixed into a base material, preferably plastic, of the at least one component. Advantageously, the self-sterilizing function can thus already be an inherent function of the at least one component. Even when the surface is abraded by mechanical wear, the at least one component can continue to be photoactivatable. It is also possible, for example, to dispense with coatings with the photoactivatable material, at least in the original state.

In another embodiment, the container processing system also has an inspection device. The inspection device is configured to inspect the containers for contamination and/or a germ load (e.g., optically, physically and/or chemically). The inspection device can also be configured to effect an output at a user interface of the container processing system and/or a remote control center when contamination and/or a germ load is detected, said output indicating that a coating having a material that can be photoactivated for the disinfecting (e.g., killing or neutralizing of germs, such as bacteria, viruses, spores, fungi) of the surface must be applied to, preferably sprayed onto, the surface. Advantageously, it can thus be ensured that, when the effect of the disinfection diminishes, e.g., due to abrasion of the surface, the surface is renewed by means of the coating, so that sufficient self-sterilization can continue to take place.

The inspection device can preferably be arranged, for example, in a measuring laboratory in order to inspect removed containers. Alternatively, the inspection device can, for example, be directly integrated in the container processing system in order to inspect the containers during operation of the container processing system.

In another embodiment, the container processing system also has an evaluation device. The evaluation device is configured to ascertain an amount of time since a last coating of the surface, to ascertain an operating duration of the container processing system and/or to count a number of containers processed by the container processing system. The container processing system can preferably also be configured to effect an output at a user interface of the container processing system and/or a remote control center when the ascertained amount of time, the ascertained operating duration and/or the counted number exceeds or reaches a predefined limit value, said output indicating that a coating having a material that can be photoactivated for the disinfecting (e.g., killing or neutralizing of germs, such as bacteria, viruses, spores, fungi) of the surface must be applied to, preferably sprayed onto, the surface. Advantageously, it can thus likewise be ensured that, when the effect of the disinfection diminishes, e.g., due to abrasion of the surface, the surface is renewed by means of the coating, so that sufficient self-sterilization can continue to take place.

The container processing system can preferably be configured for processing (e.g., producing, cleaning, coating, inspecting, filling, closing, labeling, printing on, and/or packaging) containers for liquid media, preferably beverages or liquid foods. For example, the container processing system can have a filling device for filling the containers with a liquid or pasty filling medium and/or a closing device for closing the filled containers.

For example, the containers can be configured as bottles, cans, canisters, cartons, vials, etc.

Preferably, the term “evaluation unit” can refer to electronics (e.g., with microprocessor(s) and data memory) which can take on processing tasks. The evaluation unit can, for example, be part of a control system of the container processing system.

Another aspect of the present disclosure relates to a method for treating a container processing system as disclosed herein. The method comprises disinfecting the surface of the at least one component by irradiating the material of the surface with light. Advantageously, the same advantages already described for the container processing system can be achieved with the method.

In one exemplary embodiment, the method also comprises, upon occurrence of at least one predefined event:
requesting that a user apply a coating, which has a material that can be photoactivated for the disinfecting of the surface, to the surface; and/or
applying a coating, which has a material that can be photoactivated for the disinfecting of the surface, to the surface.

In another exemplary embodiment, the at least one predefined event comprises:
sensing (e.g., by means of an inspection apparatus) contamination and/or a germ load of a container (e.g., already partially or completely processed or still to be processed by the container processing system); and/or
elapsing of a predefined operating duration of the container processing system (e.g., ascertained by an evaluation device, e.g., of the container processing system or of a remote control center); and/or
reaching a predefined number of containers processed by the container processing system (e.g., counted by an evaluation device, e.g., of the container processing system or of a remote control center); and/or
elapsing of a predefined amount of time, preferably since a last coating of the surface (e.g., ascertained by an evaluation device, e.g., of the container processing system or of a remote control center).

The preferred embodiments and features of the invention described above can be combined with one another as desired.

### BRIEF DESCRIPTION OF THE DRAWINGS

Further details and advantages of the invention are described below with reference to the accompanying drawings. In the figures:

FIG. 1 shows a schematic representation of a container processing system according to an exemplary embodiment of the present disclosure; and

FIG. 2 a schematic sectional view of components of a container processing system and of a container.

### DETAILED DESCRIPTION OF EMBODIMENTS

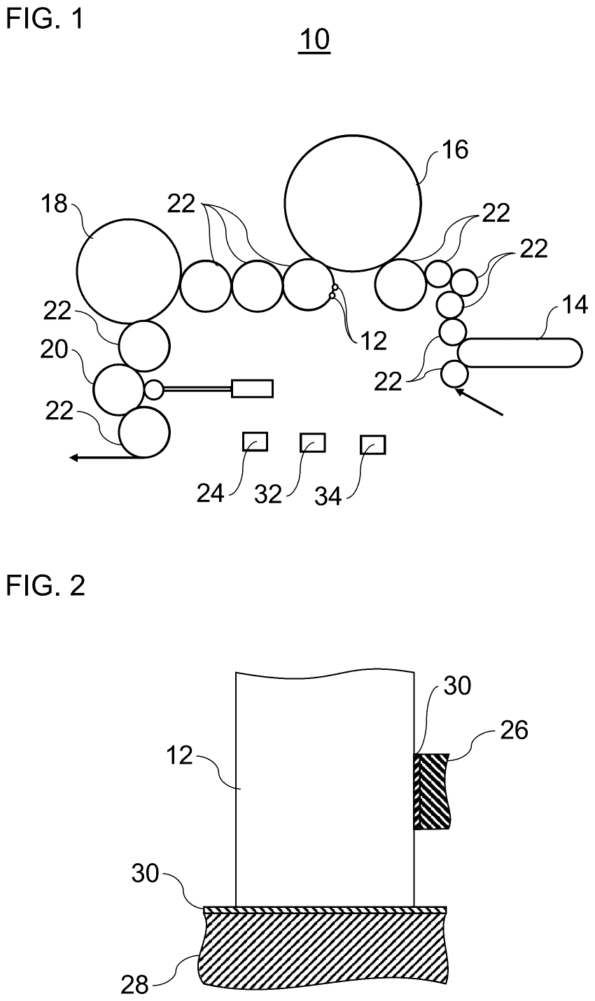

FIG. 1 shows a container processing system 10 for processing containers 12, of which only two are shown, by way of example, in FIG. 1.

For example, the container processing system 10 can produce, clean, inspect, fill, close, label, print on, group, and/or package the containers 12.

The containers 12 can be, for example, bottles, cans, canisters, cartons, flacons, etc. The containers are preferably used to hold liquid or pasty media. The containers 12 are preferably used to hold beverages or foods.

The container processing system 10 has at least one container processing device 14, 16, 18 and/or 20 and/or at least one container conveyor 22. The container processing system 10 can also have a user interface 24.

The container processing device 14 in the form of a heating apparatus can heat preforms (container blanks) to a desired temperature.

The container processing device 16 in the form of a container production device can produce the containers 12 from preforms. For example, the container production device can be configured as a container blow-molding device, preferably a stretch blow-molding device, for the blow molding of the containers from the preforms. The container production device is preferably configured as a container production carousel. The container production device can have a plurality of production stations, e.g., blow-molding stations, for simultaneously producing a plurality of containers 12. For example, the production stations can be arranged around a circumference of the container production device configured as a container production carousel. The container production device can be arranged downstream of the heating apparatus with respect to a container stream.

The container processing device 18 in the form of a filling device can fill the containers 12, preferably with a liquid or pasty medium. The filling device is preferably configured as a filler carousel. The filling device can have a plurality of filling valves for simultaneously filling a plurality of containers 12. For example, the filling valves can be arranged around a circumference of the filling device configured as a filler carousel. The filling device can be arranged, with respect to a container stream, downstream of the container production device, if present.

The container processing device 18 in the form of a closing device can close the containers 12, e.g., with a lid, a cork, a crown cap, or a screw closure. The closing device can preferably be configured as a closer carousel. The closing device can have a plurality of closing stations for simultaneously closing a plurality of containers. For example, the closing stations can be arranged around a circumference of the closing device configured as a closer carousel. The closing device can be arranged downstream of the filling device with respect to a container stream.

It is possible for the container processing system 10 to have at least one alternative or additional container processing device. For example, the container processing system 10 can have a coating device for (e.g., internally) coating the containers 12, a rinsing device for rinsing the containers 12, an inspection device for inspecting the containers 12, a labeling device for labeling the containers 12, a printing device for printing on the containers 12, and/or a packaging device for packaging the containers 12.

The at least one container conveyor 22 can transport the containers 12 through the container processing system 10. The at least one container conveyor 22 can connect the container processing devices (e.g., 14, 16, 18 and/or 20) of the container processing system 10 to one another. The at least one container conveyor 22 can have, for example, at least one transport starwheel and/or at least one linear conveyor, e.g., belt conveyor or mat chain conveyor (e.g., with a plastic mat chain).

The user interface 24 can be configured to receive inputs from a user and/or to output information to a user. For example, the user interface 24 can have an operating element (e.g., button, key, switch, etc.), a preferably touch-sensitive display, a keyboard, an emergency-off button, a safety switch, and/or a door opener.

A special feature of the present disclosure is that it is proposed that a surface of at least one component of the container processing system 10 has a material that can be photoactivated for the disinfecting of the surface.

The material is preferably a photocatalyst. For example, the material comprises titanium dioxide. For example, the material can, when irradiated with light, be stimulated to transfer energy to oxygen in the surroundings of the surface, so that singlet oxygen is formed for disinfecting the surface. The material can preferably absorb light in the spectral range of 400 nm to 700 nm. The extent of the singlet oxygen can be, for example, between 1 mm and 5 mm. A singlet oxygen layer adjoining the surface can be formed. The singlet oxygen layer can, for example, have a thickness or height between 1 mm and 5 mm. The singlet oxygen layer can continuously disinfect the surface. The singlet oxygen is an oxidizing agent that can neutralize germs by oxidative degeneration.

The at least one component can be any component of the container processing system 10. For example, the at least one component can comprise the container processing device 14, 16, 18 and/or 20. Alternatively or additionally, the at least one component can comprise, for example, at least one container conveyor 22. Alternatively or additionally, the at least one component can comprise, for example, the user interface 24.

The at least one component can preferably be arranged upstream of the filling device in order to support hygienic filling and, for example, to prevent recontamination downstream of a rinsing device (not shown). For example, the at least one component can be arranged in a portion between a sweep-off device and an entrance of the filling device or in a portion between an exit of the rinsing device and an entrance of the filling device.

The surface can preferably be a surface which is frequently touched by hand, for example a surface of the user interface 24, or a contact surface which touches the containers 12 during operation of the container processing system 10. An exemplary embodiment in which the surface provided with a photoactivatable material is part of a guide element and/or part of a transport element of the container processing system 10 is explained below with reference to FIG. 2.

FIG. 2 shows, purely schematically, a portion of the container processing system 10, in which portion the containers 12 are moved or transported. The portion can be comprised, for example, in one of the container conveyors 22 and/or in one of the container processing devices 14, 16, 18 and/or 20.

The portion can have a guide element 26 for guiding the containers 12 and/or a transport element 28 for transporting the containers 12.

The guide element 26 can guide the containers 12 during transport, preferably laterally. The guide element 26 can move along with the containers 12 or the guide element can be stationary or not move along with the containers 12 during transport.

The guide element 26 can be configured, for example, as a rod-shaped guide railing or a lateral guide wall. Alternatively, the guide element 26 can, for example, be configured as a wear strip of a guide device. Alternatively, the guide element 26 can be configured, for example, as a format part or set part which is adapted to a particular format or geometry of the containers 12 and which, for example, can be exchanged when the format is changed. For example, the format part can have recesses in the shape of cylindrical shell segments for contacting cylindrical-shell-shaped wall portions of the containers 12. For example, the format part can be comprised in a rotary machine, such as a transport starwheel.

It is possible that an additional guide element (not shown) for laterally guiding the containers 12 is provided opposite the guide element 26. The guide element 26 and the additional guide element can guide the containers 12 between them. The additional guide element can, for example, be configured like the guide element 26.

The transport element 28 can transport the containers 12. The transport element 28 can move along with the containers 12 during transport or can move the containers 12 in the transport direction. The transport element 28 can be a circulating transport element, for example.

For transport, the transport element 28 can support the containers 12 at the bottom of the containers, for example, as shown in FIG. 2. Accordingly, the transport element 28 can be configured, for example, as a transport chain, a conveyor belt or a transport mat (e.g., transport mat chain, such as a plastic mat chain).

It is also possible for the transport element 28 to support the containers 12, for example, at a container wall, a container neck and/or a container neck ring for transport. Accordingly, the transport element 28 can be configured, for example, as an active or passive container clamp or container gripper (not shown).

The guide element 26 and/or the transport element 28 can be coated with a coating 30. The coating 30 can comprise the photoactivatable material, for example titanium dioxide. The coating 30 forms a surface of the guide element 26 or of the transport element 28.

The coating 30 can be applied in any suitable manner. Coating 30 is preferably spray-applied or sprayed on. Alternatively, the coating can be, for example, spread on or applied by means of an additive manufacturing method.

Even if, with reference to FIG. 2, only one exemplary embodiment is described in which the guide element 26 and the transport element 28 have the coating 30, the present disclosure is not limited thereto. Any component of the container processing system, in particular those that were described herein with reference to FIG. 1, can have the coating 30.

As an alternative to the coating 30, it is also possible, for example, for the photoactivatable material to be mixed into a base material, preferably plastic, of the at least one component in order to disinfect the surface. This technique can again be applied to any component of the container processing system 10, in particular to those that were described herein with reference to FIGS. 1 and 2.

The photoactivatable material can already be included during the primary shaping of the at least one component. For example, the at least one component can be produced by means of an additive manufacturing method (e.g., 3D printing), e.g., from a base material (e.g., a plastic) with the photoactivatable material as an admixture or additive material. The additive manufacturing can result in a layered structure of the at least one component. It is possible for the at least one component to be additively manufactured in such a way that the concentration of the photoactivatable material is highest in a layer of the at least one component near the surface and/or is higher in said layer than in a layer of the at least one component remote from the surface. It is possible for the photoactivatable material to be comprised only in at least one layer of the at least one component near the surface. It is also possible for the photoactivatable material to be included directly in the production of the at least one component in another suitable forming method.

It is possible for the application of the coating 30 to be repeated at certain intervals. Reapplication of the coating 30 can be triggered, for example, by various predefined events.

For example, the containers 12 can be examined for contamination and/or a germ load by means of at least one inspection device 32. For example, the examination can relate to an outer wall and/or an inner volume of the containers 12. If contamination and/or a germ load is detected which, for example, is above a predefined limit value, an output specifying or indicating that the application of a coating 30 is required can be generated or brought about.

The inspection device 32 can be integrated in the container processing system 10, for example at an entrance of the filling device or at any other position. It is also possible for a container 12 to be removed from a container stream of the container processing system 10, for example manually, and transported to the inspection device 32, for example in a measuring laboratory of the factory.

For example, operation of the container processing system can be monitored by an evaluation device 34. The evaluation device 34 can be part of the container processing system 10 or be spatially associated therewith. However, it is also possible for the evaluation device 34 to be part of a remote control center of the manufacturer of the container processing system 10, i.e., to be part of the container processing system 10 but to be remote therefrom.

The evaluation device 34 can, for example, ascertain an amount of time since the application of the last coating 30 of the surface, ascertain an operating duration of the container processing system 10 and/or count a number of containers 12 processed by the container processing system 10. When the ascertained amount of time, the ascertained operating duration and/or the counted number of containers exceeds or reaches a predefined limit value, an output specifying or indicating that the application of a coating 30 is required can be generated or brought about.

The output brought about or generated by the inspection device 32 and/or by the evaluation device 34 can occur, for example, at the user interface 24. Alternatively or additionally, the output can occur, for example, at a remote or distant control center of a manufacturer of the container processing system 10. On the basis of the output, the application of the coating 30 can then be ordered, for example.

The invention is not limited to the preferred exemplary embodiments described above. Rather, a plurality of variants and modifications is possible which likewise make use of the inventive concept and therefore fall within the scope of protection. In particular, the invention also claims protection for the subject matter and the features of the dependent claims, irrespective of the claims to which they refer. In particular, the individual features of independent claim 1 are each disclosed independently of one another. In addition, the features of the sub-claims are also disclosed independently of all the features of independent claim 1. All ranges specified herein are to be understood as disclosed in such a way that all values falling within the respective range are individually disclosed, e.g., also as the respective preferred narrower outer limits of the respective range.

### LIST OF REFERENCE SIGNS

10 container processing facility
12 containers
14-20 container processing device
22 container conveyor
24 user interface
26 guide element
28 transport element
30 coating
32 inspection device
34 evaluation device

1-15. (canceled)

16. A container processing system for processing containers, comprising:
at least one component having a surface with a material that can be photoactivated for the disinfecting of the surface.

17. The container processing system according to claim 16, wherein at least one of:
the material is a photocatalyst material and the material comprises titanium dioxide.

18. The container processing system according to claim 16, wherein at least one of:
the material, when irradiated with light, is stimulated to transfer energy to oxygen in the surroundings of the surface, so that singlet oxygen is formed for disinfecting the surface; and
the material, when irradiated with light, forms a singlet oxygen layer for disinfecting the surface.

19. The container processing system according to claim 16, wherein:
the at least one component has at least one of a user interface, an operating element, a touch-sensitive display, a keyboard, an emergency-off button, a safety switch, and a door opener.

20. The container processing system according to claim 16, wherein:
the surface of the at least one component is a container contact surface for contacting containers during operation of the container processing system.

21. The container processing system according to claim 16, wherein the at least one component comprises:
a guide element for guiding the containers.

22. The container processing system according to claim 21, wherein the guide element includes one of:
a guide railing, a guide wall, a guide format part adapted to a geometry of the containers to be processed, and a wear strip of a container guide.

23. The container processing system according to claim 16, wherein at least one of:
the at least one component comprises a transport element for transporting the containers; and
the at least one component comprises a container conveyor for conveying the containers; and
the at least one component is arranged in a portion of the container processing system between a rinsing device of the container processing system and a filling device of the container processing system.

24. The container processing system according to claim 23, wherein at least one of:
the transport element includes one of a transport chain, a conveyor belt, a transport mat, a container clamp, and a container gripper; and
the container conveyor includes one of a transport starwheel and a linear conveyor.

25. The container processing system according to claim 16, wherein:
the at least one component comprises a container processing device for processing the containers.

26. The container processing system according to claim 25, wherein the container processing device includes one of:
a heating device for heating preforms for producing the containers;
a container production device for producing the containers;
a coating device for coating the containers;
a rinsing device for rinsing the containers;
a filling device for filling the containers;
a closing device for closing the containers;
an inspection device for inspecting the containers;
a labeling device for labeling the containers;
a printing device for printing on the containers; and
a packaging device for packaging the containers.

27. The container processing system according to claim 16, wherein:
the surface is formed by a spray-applied coating having the material.

28. The container processing system according to claim 16, wherein:
the photoactivatable material is mixed into a base material of the at least one component.

29. The container processing system according to claim 28, wherein the base material includes plastic.

30. The container processing system according to claim 16, further comprising:
an inspection device configured to:
inspect the containers for at least one of contamination and a germ load; and
effect an output at a user interface of at least one of the container processing system and a remote control center when at least one of contamination and a germ load is detected, said output indicating that a coating having a material that can be photoactivated for the disinfecting of the surface must be applied to the surface.

31. The container processing system according to claim 16, further comprising:
an evaluation device configured to:
at least one of ascertain an amount of time since a last coating of the surface, ascertain an operating duration of the container processing system and count a number of containers processed by the container processing system; and
at least one of effect an output at a user interface of the container processing system and a remote control center when the ascertained amount of time, the ascertained operating duration and the counted number exceeds or reaches a predefined limit value, said output indicating that a coating having a material that can be photoactivated for the disinfecting of the surface must be applied to the surface.

32. A method for treating a container processing system according to claim 16, comprising:
disinfecting the surface of the at least one component effected by irradiating the material of the surface with light.

33. The method according to claim 32, further comprising:
upon occurrence of at least one predefined event, at least one of:
requesting that a user apply a coating, which has a material that can be photoactivated for the disinfecting of the surface, to the surface; and
applying a coating, which has a material that can be photoactivated for the disinfecting of the surface, to the surface.

34. The method according to claim 33, wherein the at least one predefined event comprises at least one of:
sensing at least one of contamination and a germ load of a container; and
elapsing of a predefined operating duration of the container processing system; and
reaching a predefined number of containers processed by the container processing system; and
elapsing of a predefined amount of time.

35. The method according to claim 34, wherein the predefined amount of time is since a last coating of the surface.
